(19) United States
(12) Patent Application Publication
(10) Pub. No.: US 2025/0082497 A1
(43) Pub. Date: Mar. 13, 2025

(54) THERAPEUTIC BELT WITH POCKETS AND BALLS

(71) Applicant: Bryan Okpala, St. Louis, MO (US)

(72) Inventor: Bryan Okpala, St. Louis, MO (US)

(21) Appl. No.: 18/957,080

(22) Filed: Nov. 22, 2024

Related U.S. Application Data
(60) Provisional application No. 63/669,184, filed on Jul. 9, 2024.

(51) Int. Cl. A61F 7/02; A61F 7/00

(52) U.S. Cl. CPC A61F 7/02; A61F 2007/0027; A61F 2007/0231; A61F 2007/0268; A61F 2007/0295

(57) ABSTRACT

The present invention is directed to a pain-relieving treatment belt that comprises a plurality of pockets positioned on its interior surface which target the vertebrae of the lumbar spine and house a plurality of balls comprised of a thermally conductive material. The balls are designed to provide heat or cold therapy, as well as pressure and massage to the lumbar spine. The balls may be heated or cooled, and movable, to target specific spinal discs and muscles in the lumbar region. The belt is designed to rehabilitate the lower back by promoting circulation and alleviating inflammation and other ailments through the use of heated or cooled balls that rotate and apply pressure to targeted parts of the lower back.

### CROSS REFERENCE TO RELATED APPLICATIONS

This application claims the benefit of priority to U.S. Provisional Application No. 63/669,184, filed on Jul. 9, 2024.

### BACKGROUND

### 1. Field of the Invention

The present invention relates generally to a belt for pain relief and rehabilitation of the lower back.

### 2. Description of Related Art

The application of heat and cold to the skin have been traditionally used to relieve pain or discomfort resulting from various acute and chronic muscle, spinal, and myofascial conditions. Additionally, some devices have included balls or other objects which perform a massaging function and soothe muscle pain or discomfort.

So as to reduce the complexity and length of the Detailed Specification, and to fully establish the state of the art in certain areas of technology, Applicant herein expressly incorporates by reference all of the following materials identified in each numbered paragraph below.

US20090192423A1 discloses a therapeutic massage device containing a plurality of pockets along its length designed to receive a plurality of balls.

US20030176264A1 discloses a flexible fitness and rehabilitation exercising and massaging apparatus that incorporates a plurality of internal pockets that securely hold removable objects.

U.S. Pat. No. 9,962,284B2 discloses a reusable pain treatment device that comprises one or more conductive members extending from the inner surface of the device that are capable of transferring heat, cold or vibrations from disposable or reusable packs.

U.S. Pat. No. 11,648,173B2 discloses an elongated tubular sleeve with massage ball receiving cavities between the ends of the sleeve, and at least one massage ball received within the massage ball receiving cavity.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57 (c) (1)-(3), Applicant(s) will amend the specification to expressly recite the essential material that is incorporated by reference as allowed by the applicable rules.

### SUMMARY

Implementations of a therapeutic belt device may comprise a plurality of pockets containing balls comprised of a thermally conductive material with such balls specifically targeting the vertebrae of the lumbar spine.

Implementations of a therapeutic belt device may comprise a strap with a plurality of thermally conductive balls housed in a plurality of pockets, the pockets positioned to align with lumbar vertebrae.

Particular aspects may comprise one or more of the following features. The pockets may be aligned to target specifically the L3, L4 and L5 vertebrae of the lumbar spine. The pockets may in some implementations be arranged into a diamond or rhomboid, or into vertically arranged columns. Balls may be comprised of any suitable material including, but not limited to, rubber, neoprene, vinyl, foam, or leather. The pockets may be comprised of any suitable material including, but not limited to, fabric, vinyl, or leather. The therapeutic belt may, in some arrangements, be four to six inches in width. The thermally conductive balls may, in some arrangements, be one to two-and-a-half inches in diameter. The therapeutic belt may be adapted to a hook-and-loop fastener system, a buckle system, or a magnetic clasp system.

Implementations of a method for rehabilitating a lumbar region of a user may comprise one or more of the following steps: heating two thermally conductive balls, in some implementations to a range of about 22 degrees Celsius to about 45 degrees Celsius, or cooling two or more thermally conductive balls, in some implementations to a range of about 0 degrees Celsius to about 21.5 degrees Celsius. The balls may be placed in two pockets situated on the inner surface of the therapeutic belt, wherein the pockets are situated to target at least two vertebrae of the lumbar spine of the user, and may more specifically target the L3, L4, and L5 vertebrae. The therapeutic belt may be fastened around the user such that the therapeutic balls apply pressure to the spine of the user and rotate on their center in response to user movement. The location of the pockets on the interior surface of the belt may be movable and may be moved to target at least one of the L3, L4, and L5 vertebrae.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographers, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of 35 U.S.C. § 112 (f). Thus, the use of the words “function,” “means” or “step” in the Detailed Description or Description of the Drawings or Claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION, DRAWINGS, and CLAIMS.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a side view of a therapeutic belt in use.

FIG. 2 depicts a front view of the therapeutic belt according to a first embodiment.

FIG. 3 depicts a front view of the therapeutic belt according to a second embodiment.

FIG. 4 depicts a ball transferring heat or cold when pressure is applied.

FIG. 5 depicts a perspective view of the therapeutic belt according to the first embodiment of FIG. 2.

FIG. 6 depicts a cross-sectional view of an implementation of the therapeutic belt with two pockets and a ball housed in each pocket.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the invention is not limited to the examples that are described below.

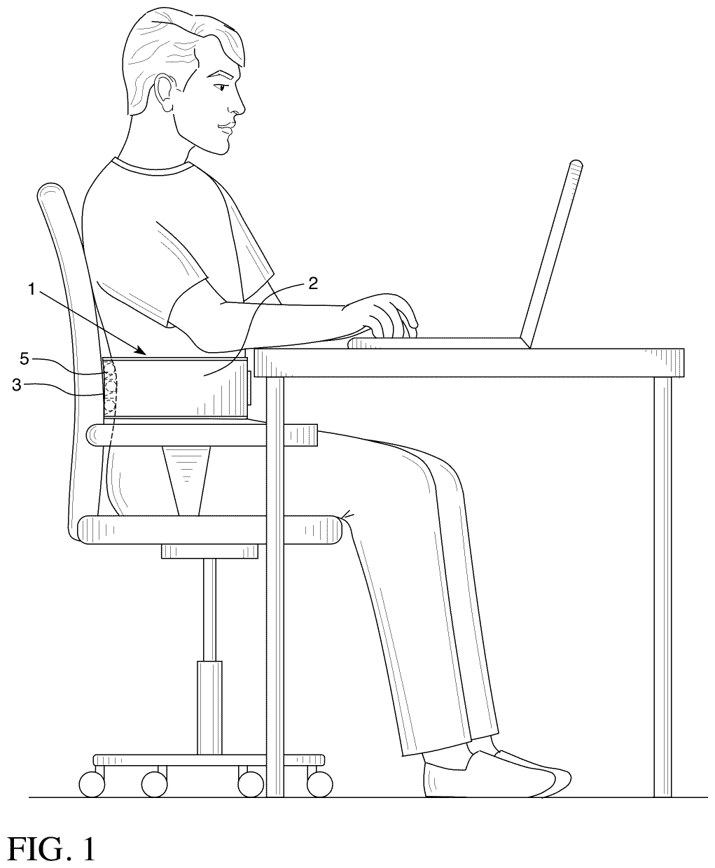

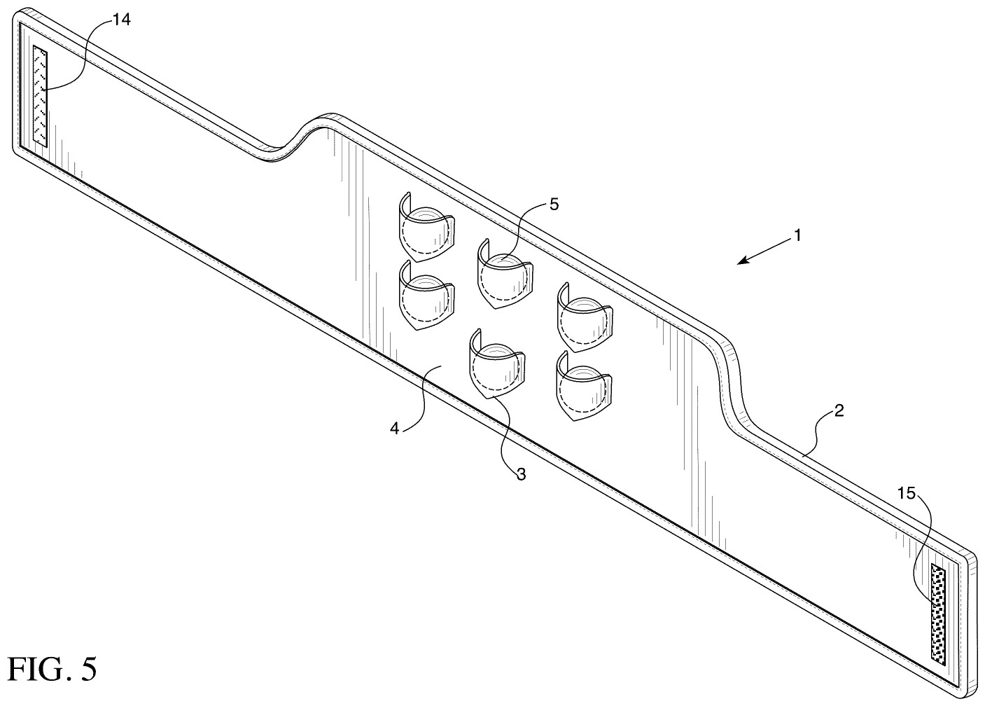

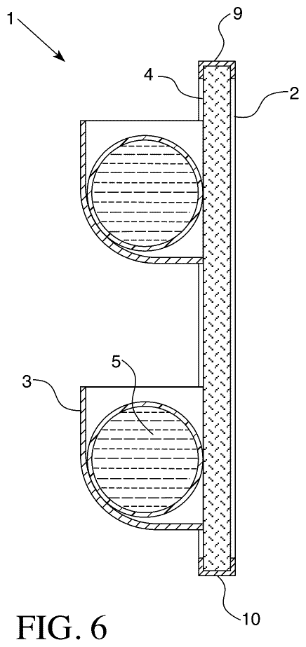

FIG. 1 depicts an exemplary embodiment of a therapeutic belt 1 in use on the lumbar region of a user. As shown in FIG. 5, in some embodiments the therapeutic belt 1 may comprise a strap 2 with a plurality of pockets 3 located on an inner surface 4, and a plurality of thermally conductive balls 5 situated such that at least one of the pockets among the plurality of pockets is configured to house a thermally conductive ball from among the plurality of thermally conductive balls.

The strap 2 may be constructed from any suitable textile including, but not limited to, neoprene, leather, Lycra, or spandex. The material of strap 2 may be breathable and elastic for comfort and flexibility. Various embodiments of the therapeutic belt 1 may comprise various numbers and spatial arrangements of the pockets 3. The therapeutic belt 1 may be as wide or as long as needed for wearability, and in one embodiment may be about 4 to 6 inches wide, within a reasonable tolerance based on standard manufacturing methods of the given material. With regard to the width of the therapeutic belt 1, the term “about” is intended to mean within one inch wider or narrower than the specified width range. The length of the therapeutic belt 1 may be adjustable to fit various waist sizes. The average length of a male's spinal column is about 71 centimeters. The cervical part measures 12.5 centimeters, the lumbar 18 centimeters, and the sacrum and coccyx 12.5 centimeters. In some embodiments, the length and width of the belt may be in line with the aforementioned measurements. The therapeutic balls 5 may be of various sizes depending in part on the arrangement of the pockets, target areas, or user need. In one embodiment, the therapeutic balls 5 are about 1 to 2.5 inches in diameter, within a reasonable tolerance based on standard manufacturing methods of the given material. With regard to the width of the diameter of the therapeutic balls 5, the term “about” is intended to mean within one half inch greater or smaller than the specified diameter range.

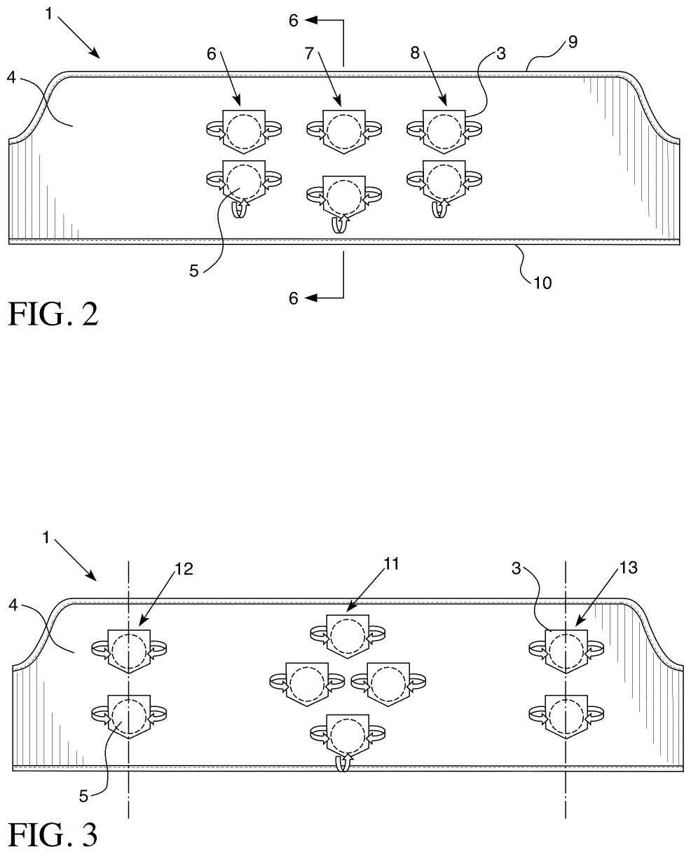

Referring to FIG. 2, in one embodiment the plurality of pockets 3 is arranged into three columns 6, 7, 8 wherein the first 6 and third 8 columns begin at a point proximal to an upper edge 9 of the strap, and the second column 7 is positioned proximal to a lower edge 10 of the strap. The positioning of the proximally located pockets 3 and balls 5 are to provide support, pressure, massage, and heat or cold therapy to the L3 to L5 lumbar vertebrae of the user.

Referring to FIG. 3, another embodiment of the present invention is depicted wherein the plurality of pockets 3 and plurality of balls 5 are located in three areas. A first plurality 11 of four pockets are located centrally on the inner surface 4 of the strap, each of the four pockets positioned at a different vertex of a diamond. A second plurality of pockets 12 located on a first side of the inner surface 4 of the strap and located coaxially on a longitudinal axis running perpendicular to an upper edge 9 and a lower edge 10 of the strap. A third plurality of pockets 13 are located on a second side of the inner 4 surface of the strap and located coaxially on a longitudinal axis running perpendicular to an upper edge 9 and a lower edge 10 of the strap. The positioning of the centrally, first-side and second side located pockets 3 and balls 5 are to provide support, pressure, massage, and heat or cold therapy to the L3 to L5 lumbar vertebrae and oblique muscles of the user.

While any appropriate closure method is contemplated by the present disclosure, in some embodiments, the therapeutic belt 1 may comprise a first strip 14 and a second strip 15 of at least one of a buckle, a hook and loop fastener system, or a magnetic clasp system.

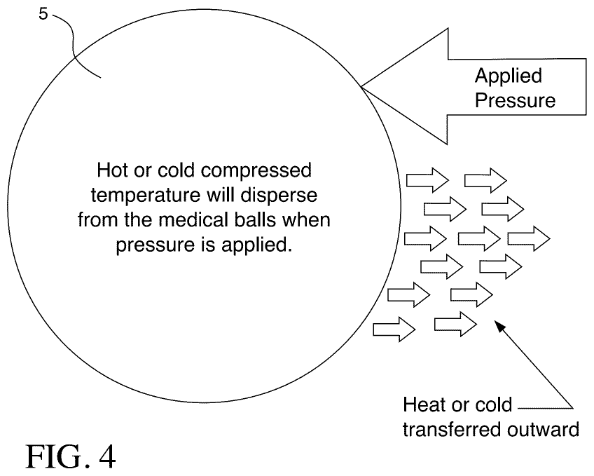

Referring to FIG. 4, the therapeutic ball 5 is depicted transferring heat or cold in response to pressure applied by the user in the course of regular use. In FIG. 4 the therapeutic ball 5 is also depicted transferring heat or cold outward to provide hot or cold therapy to the user when the therapeutic belt 1 is in use. The ball 5 can be constructed from any suitable material including, but not limited to, rubber, vinyl, neoprene, polystyrene foam, or leather. In one embodiment, the ball 5 may be comprised of thermally insulating material, or covered in thermally insulating material, including thermal roll. In some embodiments, the balls 5 can be padded with soft, hypoallergenic foam to ensure comfortable yet rehabilitating pressure on the user's back. In some embodiments, the balls 5 may be lined with rubber to increase durability.

Referring to FIGS. 2-3, the plurality of balls 5 may be housed in the plurality of pockets 3 in a way that allows a ball from among the plurality of balls 5 to rotate along its center in relation to the user's body movement.

In one embodiment the therapeutic belt 1 may further comprise at least one sensor that monitors the spine of the user, that detects spinal alignment and posture and delivers the information to the user through a mobile app connected to the sensor. The object of the sensor is to provide personalized suggestions to the user in improving their overall spinal health.

A purpose of the therapeutic belt 1, generally, is to provide support and rehabilitation to the lumbar region and to aid in keeping the spine properly aligned. More specifically, the therapeutic belt 1, in part, aims to reduce muscle tension by stimulating the sensory pathways of the user by diverting focus to the sensation of the balls 5. The therapeutic belt 1 aims to reduce inflammation and relax tight muscles through pressure and massage. When the user places their back against the surface area of the therapeutic belt 1, the balls 5 will rotate and disperse compressed air to the user. In an exemplary embodiment, the therapeutic belt 1 may be comfortable, adjustable, and provide adequate pressure to the user.

In some embodiments, additional straps can be added to the therapeutic belt 1 to secure the belt around the user. In an exemplary embodiment, the therapeutic belt 1 may be visually discreet, with a sleek and minimalistic design to allow for wear under clothing. The therapeutic belt 1 may be breathable and lightweight, and contour to the natural shape of the user's body.

In an exemplary embodiment of a method for rehabilitating the lumbar region of the user, the therapeutic belt 1 is fastened around the lower back of a user. FIG. 1 depicts a plurality of pockets 3 housing a plurality of balls 5, wherein the plurality of balls applies pressure to the lower back of the user and rotates in response to the movement of the user. The plurality of pockets 3 may be positioned to target at least two of the L3, L4, and L5 vertebrae.

In one embodiment the therapeutic balls 5 are cooled in a freezer to a temperature range of about 0 degrees Celsius to about 21.5 degrees Celsius. With regard to the temperature of the balls 5, the term “about” is intended to mean within one degree Celsius greater or smaller than the specified temperature range. The cooled balls may, in part, reduce swelling, nerve activity, and inflammation, and provide relief from acute pain.

In one embodiment the therapeutic balls 5 are heated in a microwave to a temperature range of about 22 degrees Celsius to about 45 degrees Celsius. With regard to the temperature of the balls 5, the term “about” is intended to mean within one degree Celsius greater or smaller than the specified temperature range. The heated balls may, in part, promote muscle relaxation, and increase blood flow to the contact areas.

In one embodiment the location of the plurality of pockets 3 may be movable and may be movable to target at least one of the L3, L4 and L5 vertebrae.

In an exemplary embodiment, the therapeutic belt 1 can be used on a yoga mat or a chair. The therapeutic belt 1 may, in some embodiments, be suitable for use in bed, and keep the spine supported as the user moves during sleep. The therapeutic belt 1 may be used by sedentary users to support their lower backs by causing a slight arch in the back that prevents slumping, aligns the spine, and allows the users to move more freely. The user wearing the therapeutic belt 1 may push it against the wall, floor, yoga mat, chair, or bed, and move their body in a circular motion to apply pressure and massage to the lower back. The therapeutic belt 1 may be used during rehabilitative exercises to improve muscle strength and flexibility.

The therapeutic belt 1 may be used by military personnel and athletes recovering from injuries, office workers and truck drivers suffering from chronic back pain due to sitting, elderly individuals afflicted by stiffness or joint pain, healthcare professionals seeking solutions for patients, and patients in physical therapy or rehabilitation programs.

In places where the description above refers to particular implementations of the device and methods for a therapeutic belt it should be readily apparent that a number of modifications may be made without departing from the spirit thereof and that these implementations may be applied to other devices and methods for a therapeutic belt.

I claim:

1. A therapeutic belt comprising:
a plurality of pockets positioned on an inner surface of the therapeutic belt; and
a plurality of balls, the balls comprised of a thermally conductive material, wherein at least two pockets among the plurality of pockets are positioned such that a ball housed within each of the at least two pockets align with at least two vertebrae of a lumbar spine of the user.

2. The therapeutic belt of claim 1 wherein the at least two vertebrae comprise at least two of the L3, L4, and L5 vertebrae of the lumbar spine.

3. The therapeutic belt of claim 1 further comprising at least four pockets, each of the at least four pockets positioned at a different vertex of at least one of a diamond and a rhombus.

4. The therapeutic belt of claim 1 wherein the at least two pockets comprise a plurality of vertically arranged columns.

5. The therapeutic belt of claim 1 wherein the at least two balls are comprised of one or more of a rubber, a neoprene, a vinyl, a foam, and a leather.

6. The therapeutic belt of claim 1 wherein the at least two pockets are comprised of one or more of a fabric, a vinyl, and a leather.

7. The therapeutic belt of claim 1 wherein a width of the therapeutic belt is within a range of about 4 inches to about 6 inches.

8. The therapeutic belt of claim 1 wherein a diameter of each of the at least two balls is within a range of about 1 inch to about 2.5 inches.

9. The therapeutic belt of claim 1 further comprising a first fastener strip and a second fastener strip, wherein the first fastener strip is a hook and loop closure strip coupled to a first end of the therapeutic belt and the second fastener strip is a hook and loop strip coupled to a second end of the therapeutic belt, the first and second hook and loop strips being complementary to one another to enable closure of the therapeutic belt around a user.

10. The therapeutic belt of claim 1 further comprising a first fastener strip and a second fastener strip, wherein the first fastener strip is a magnetic strip coupled to a first end of the therapeutic belt and the second fastener strip is a magnetic strip coupled to a second end of the therapeutic belt, the first and second magnetic strips being complementary to one another to enable closure of the therapeutic belt around a user.

11. A method for rehabilitating a lumbar region of a user comprising:
altering a temperature of at least two thermally conductive balls;
placing the at least two thermally conductive balls within at least two pockets positioned on an interior surface of a therapeutic belt, the at least two pockets positioned such that the at least two pockets align with at least two vertebrae of a lumbar spine of the user; and
securing the therapeutic belt around the lumbar region of the user.

12. The method of claim 11 wherein the at least two pockets align with at least two of the L3, L4, and L5 vertebrae of the lumbar spine.

13. The method of claim 11 further comprising heating the at least two thermally conductive balls to a therapeutically exothermic state.

14. The method of claim 11 further comprising cooling the at least two thermally conductive balls to a therapeutically endothermic state.

15. The method of claim 13 wherein the at least two thermally conductive balls are heated to a temperature within a range of 22 degrees Celsius to about 45 degrees Celsius.

16. The method of claim 14 wherein the at least two thermally conductive balls are cooled within a range of 0 degrees Celsius to about 21.5 degrees Celsius.

17. The method of claim 11 wherein the therapeutic belt is fastened around the user such that the at least two therapeutic balls apply pressure to the lumbar spine of the user.

18. The method of claim 11 wherein the at least two thermally conductive balls are situated in the at least two pockets such that the at least two thermally conductive balls rotate on their center in response to movement of the user.

19. The method of claim 11 wherein the location of pockets on the interior surface of the therapeutic belt are movable.

20. The method of claim 19 wherein the location of pockets on the interior surface are movable to align with at least one of an L3, L4, and L5 vertebrae of the lumbar spine of the user.
